(19) United States
(12) Patent Application Publication
(10) Pub. No.: US 2004/0204375 A1
(43) Pub. Date: Oct. 14, 2004

(54) Gene delivery compounds

(76) Inventors: Eric J Sorscher, Birmingham, AL (US); Jeong S Hong, Birmingham, AL (US); Jennifer E Harris, Birmingham, AL (US); Kimberly V Curlee, Phoenix, AZ (US); Joseph A Maddry, Birmingham, AL (US); William B Parker, Birmingham, AL (US); William R Wand, Mountain Brook, AL (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/476,754

(22) PCT Filed: May 2, 2002
(86) PCT No.: PCT/US02/13895

Related U.S. Application Data
(60) Provisional application No. 60/288,050, filed on May 2, 2001.

(51) Int. Cl. A61K 48/00; A61K 31/7076; A61K 31/15

(52) U.S. Cl. 514/44; 514/46; 514/639

(57) ABSTRACT

Compounds having the structure:

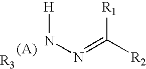

wherein R₁, R₂ and R₃ are each independently a C₀₋₁₂ substituent selected from the group consisting of: hydrogen, a heteroatom, alkyl, alkenyl, alkynyl, heteroatom substituted alkyl, heteroatom substituted alkenyl, heteroatom substituted alkynyl, aryl, arylalkyl, arylalkenyl, arylalkynyl; where the heteroatom is selected from the group consisting of: N, O and S; and where (A) is a single or double bond between N and R₃. Further the C₀₋₁₂ substituent is linear, branched or cyclic and optionally includes a pendant moiety selected from the group consisting of: carbonyl, hydroxyl, carboxyl, amine, thiol, thioester, thioether, phosphate, alkoxy, aryl, arylalkyl, sulfonamide and alkyl halide. Further, compounds 6883, 6898, 6975, 7036, 7064 and 8496 are provided. A process is provided for activating gene transfer in a subject by administering a pharmaceutically effective amount of a gene transfer activating compound to a subject and delivering pharmaceutically effective amount of a vector containing a nucleic so that the nucleic acid is transcribed in a target cell of the subject. A process for activating gene transfer to a cell is provided. A kit for activating gene transfer is provided.

### GRANT REFERENCE

The research carried out in connection with this invention was supported in part by grants NIH-NIDDK 5 P30 DK54781 and NIH NCI 5 U19 CA67763.

### FIELD OF THE INVENTION

The present invention generally relates to compounds promoting delivery of exogenous genes to cells. Specifically, the present invention relates to compounds which promote viral transduction. More particularly, the present invention relates to compounds promoting adenoviral gene transfer.

### BACKGROUND OF THE INVENTION

Gene therapy is the treatment of a pathological condition by introduction of an exogenous gene into a cell or tissue. In inherited diseases such as sickle cell anemia, a₁ antitrypsin deficiency, phenylketonuria, hemophilia and cystic fibrosis, the goal of gene therapy is to replace a missing or defective gene in order to allow a cell or tissue to function normally. Gene therapy can also be used to eliminate abnormal cells. In pathological conditions such as cancer, inflammation and autoimmunity, this technique allows for introduction of toxins which cause death of the targeted abnormal cells.

In spite of the promise of gene therapy for management of intractable disease, inefficient transfer of genes into cells has slowed progress towards the goal of routine, reproducible treatment. The refractoriness of cells and tissues in vivo is well known. The required level of gene transfer is rarely attained in current gene therapy protocols even though only a small number of cells are required to express the therapeutic gene in order to ameliorate the pathological condition (1-4). For instance, for treatment of cystic fibrosis it has been suggested that if 5% of target epithelial cells express one Cystic Fibrosis Transmembrane Conductive Regulator (CFTR) mRNA molecule per cell, the physiologic CT transport defect in the airways may be overcome. Nevertheless, it has not been possible to consistently achieve even this low level of gene transfer in vivo using conventional adenoviral or other vectors (1).

While gene transfer to cells and tissues in vivo is difficult in general, it is particularly difficult to transfer exogenous genes into epithelial cells. It is believed that this resistance reflects the barrier function of these tissues and failure of epithelial cell apical membranes to endocytose gene transfer vectors such as adenoviral particles (5, 6). In contrast to lack of endocytotic activity of epithelial apical membranes, basal membranes of epithelial cells do allow endocytosis. However, basal membranes are not accessible because of the tight junctions between cells.

Difficulty with gene transfer in epithelia using adenovirus vectors is well documented (5-7). However, such problems are not limited to viral constructs and it is reasonable to imagine that the barrier function of epithelia may substantially limit transduction efficiency with vectors unrelated to adenovirus by a similar mechanism.

A number of methods have been used, both in vitro and in vivo, to overcome cellular resistance to introduction of exogenous genes. While EDTA, EGTA, other calcium chelators, and abrasion all augment gene transfer to epithelial cells, results are still less than optimal for effective gene therapy (8-9).

The discovery of new means of facilitating gene transfer would benefit the overall field of gene therapy. Thus, if a safe transient activator of gene transfer to otherwise refractory tumors or tissues were identified, this drug could be given in combination with a gene therapy vector encoding a gene of choice.

### SUMMARY OF THE INVENTION

Compounds are provided having the structure

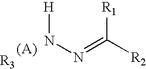

wherein R₁, R₂ and R₃ are each independently a C₀₋₁₂ substituent selected from the group consisting of: hydrogen, a heteroatom, alkyl, alkenyl, alkynyl, heteroatom substituted alkyl, heteroatom substituted alkenyl, heteroatom substituted alkynyl, aryl, arylalkyl, arylalkenyl, arylalkynyl with the proviso that at least one of R₁, R₂ and R₃ is not H; where the heteroatom is selected from the group consisting of: N, O and S; and where (A) is a single or double bond between N and R₃. Further the C₀₋₁₂ substituent is linear, branched or cyclic and optionally includes a pendant moiety selected from the group consisting of: carbonyl, hydroxyl, carboxyl, amine, thiol, thioester, thioether, phosphate, alkoxy, aryl, arylalkyl, sulfonamide and alkyl halide.

Further, compounds 6883, 6898, 6975, 7036, 7064 and 8496 are provided.

A process is provided for activating gene transfer in a subject by administering a pharmaceutically effective amount of a gene transfer activating compound to a subject and delivering a pharmaceutically effective amount of a vector containing a nucleic acid so that the nucleic acid is transcribed in a target cell of the subject.

Further described is a process for activating gene transfer to a cell.

A kit for activating gene transfer is provided.

### BRIEF DESCRIPTION OF THE DRAWINGS

FIG. 1 is a graphic illustration that compound 6975 is effective in activating adenovirus-mediated gene transfer and expression in cells wherein the arrow identifies compound efficacy.

FIG. 2 is a picture of the results of an assay showing the effectiveness of compound 6975 on adenovirus-mediated gene transfer and expression in human cervical carcinoma HeLa cells and in human colonic carcinoma HT29 cells.

FIG. 3 is a graph showing that compound 6975 activates adenoviral gene transfer of luciferase to cystic fibrosis airway epithelial IB3 cells.

FIG. 4 is a picture showing LacZ staining after adenovirus-mediated gene transfer to HT29 cells treated with EGTA or compound 6975.

FIG. 5 is a graph showing that compound 6975 activates in vivo adenoviral gene transfer to PC-3 human prostate cell tumors in mice.

FIG. 6 is a picture of the results of an assay showing that compound 8496 activates adenoviral gene transfer of LacZ to HT29 cells.

FIG. 7 is a graph showing the percent survival of HT29 and HeLa cells cultured at medium confluency treated with compounds compared to untreated cells.

FIG. 8 is a graph showing the percent survival of HT29 and HeLa cells cultured at high confluency treated with compounds compared to untreated cells.

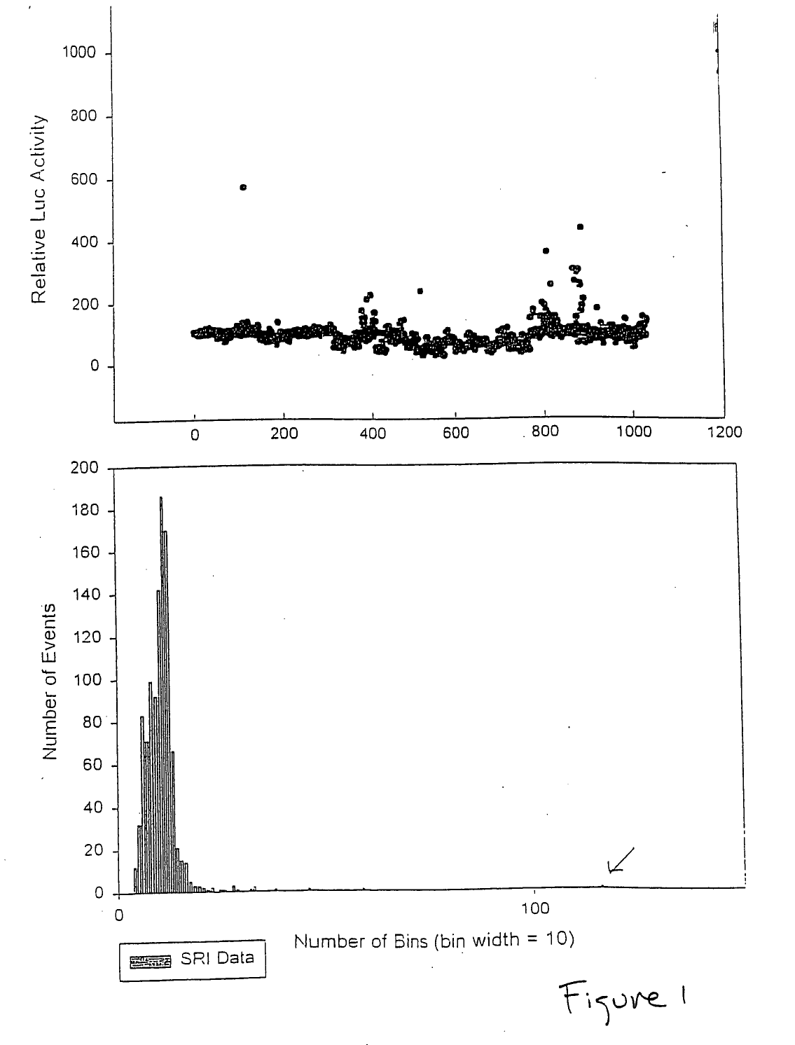

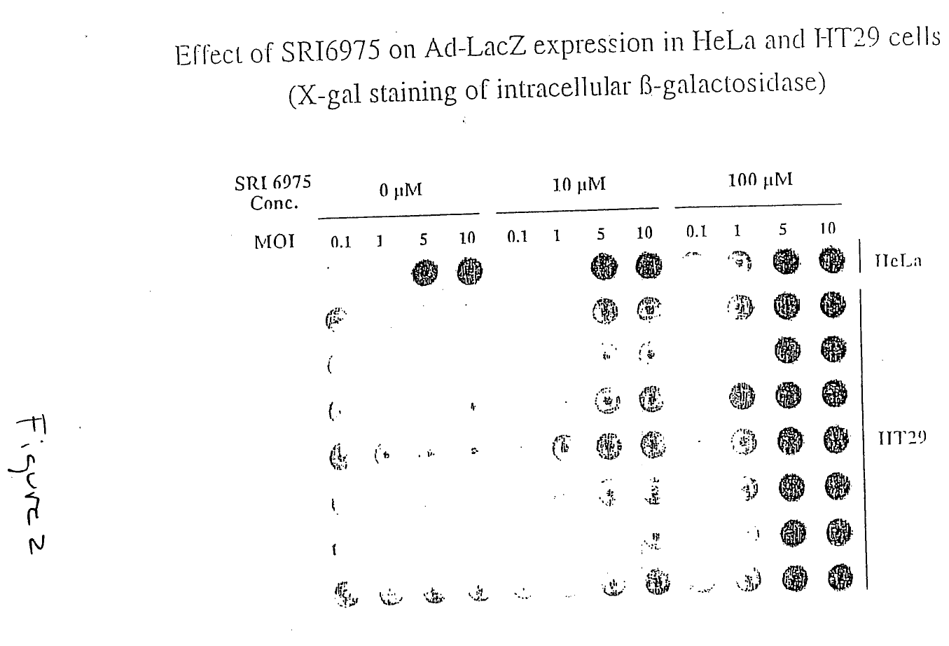

### DETAILED DESCRIPTION OF THE INVENTION

The invention provides a method for activating gene transfer by administering at least one gene transfer activating compound to a subject and delivering to the subject a vector containing a nucleic acid so that the nucleic acid is transcribed in the target cells of the subject. Thus, in one embodiment of the present invention, in a cell or tissue lacking a protein, the invention provides the cell or tissue with genetic material to synthesize the protein.

In another embodiment of the present invention, the nucleic acid encodes a protein which is a marker. The marker is an indication of a cellular function. Examples of cellular function indicated by the marker are cell uptake of the vector, transcription, translation and pH regulation. Illustrative examples of marker proteins are green fluorescent protein, luciferase and β-galactosidase.

Genetic material delivered by the process of the present invention encodes a mammalian or non-mammalian protein. Illustrative examples of non-mammalian proteins are green fluorescent protein, luciferase and β-galactosidase.

The genetic material delivered by the processes of the present invention is translated or untranslated. When the genetic material is transcribed but not translated, the transcripts affect cell function. An illustrative example of the effect of transcripts is down regulation of a targeted cellular protein due to hybridization of exogenous antisense transcripts with endogenous nucleic acids.

Delivery of the Recombinant Vector

Methods known to those skilled in the art may be used to administer the recombinant vector used in the processes of the present invention. Genetic material may be delivered to cells via viral vectors and non-viral vectors known to those skilled in the art. For example, genes may be delivered by direct injection of genetic material into tissues or cells, lipid-mediated uptake, conjugation of the gene to a protein carrier, cationic liposomes, polycationic polymer-DNA complexes and biolistic methods (1, 2, 10). However, other gene transfer methods-will also generally be applicable. Genetic material illustratively includes DNA, plasmid constructs, RNA and oligonucleotides. In a preferred embodiment, a recombinant vector includes a nucleic acid molecule encoding a protein and a promoter positioned upstream of the nucleic acid molecule. The nucleic acid is transcribed and translated in the cells to which the nucleic acid has been transferred. Among viral recombinant vectors are retroviruses (11), adenovirus (12), lentivirus (9), adeno-associated virus (14), herpes simplex virus (15) and vaccinia virus (16). A preferred embodiment of the present invention is use of compounds of the present invention to activate gene delivery using adenovirus recombinant vectors. These include both replication incompetent and permissively replicating adenoviruses.

The compounds and methods of the present invention are used to activate transfer of genetic material to subjects illustratively including human, cow, horse, sheep, pig, goat, chicken, cat, dog, mouse and rat.

The present invention provides compounds and methods useful in treatment of pathological conditions or diseases where it is desirable to introduce an exogenous protein or genetic material into the target cells or tissues of a subject. Illustrative examples of such target cells or tissues include those of the skin, nervous system, cardiovascular system, immune system, reproductive system, musculoskeletal system, lymphatic system, alimentary system, excretory system, endocrine system, hormone system and blood circulatory system. In a preferred embodiment, the present invention activates gene transfer-into an epithelial cell.

An example of the general type of pathological conditions or diseases that could be treated using the compounds and methods of the present invention is those in which at least one normal protein is lacking, either missing or produced at reduced levels. Such conditions or diseases illustratively include galactosemia, phenylketonuria, Duchenne muscular dystrophy, Lesh-Nyhan syndrome, severe combined immunodeficiency syndrome, thalassemia, sickle cell anemia, cystic fibrosis, a₁ antitrypsin deficiency, cancer, lysosomal storage disorders, porphyria and hemophilia.

In addition, the compounds and methods of the present invention are useful in treatment of conditions or diseases where it is desirable to introduce an exogenous protein which functions to reduce levels of a mutant protein. An example of the general type of pathological condition or disease where this applies is a mutation in one allele of a protein which results in a dominant phenotype which has negative effects on the cell, tissue or organism. In such a case, levels of the mutant protein may be reduced, leaving the normal protein produced by the non-mutant allele to fulfill its function. Specific elimination of an undesirable protein may be achieved by expression of specific ribozymes (17).

In addition, the compounds and methods of the present invention are useful in treatment of conditions or diseases where it is desirable to introduce an exogenous protein or genetic material which is not a normal component of the target cell or tissue. Examples include methods for treatment of growth of abnormal cells, such as cancer cells, and treatment of viral infection. In these cases, a gene is transferred to target cells to produce an enzyme which acts on co-administered agents to produce toxins that destroy the target cell.

The term “pharmaceutically active amount” as used herein is intended to mean an amount of a compound or recombinant vector that, when administered to a subject, ameliorates a symptom of the disease, disorder, or condition.

The term “abnormal cell” as used herein is intended to mean a cell which is anomalous in any of a number of ways including but not limited to: dividing in an uncontrolled manner, having chromosomal abnormalities, having a typical functional properties such as, uncontrolled release or uptake of cellular products, loss of usual contact inhibition, and unusual migratory properties. In addition, abnormal cells might be characterized by failure to mature along normal functional lines, by widely varying in size compared to a typical cell of its type and by loss of usual orientation of the cells to one another.

The term “pathogenic viral infection” as used herein is intended to mean infection by a virus causing disease or pathological effects and is distinguished from therapeutic viral infection.

The term “mutant” as used herein is intended to mean a change in a gene which has deleterious effects on the cell or organism in which the mutation occurred.

Compounds Identified as Gene Transfer Activators

A gene transfer activating compound is identified by an assay which determines whether gene transfer occurs to a greater extent in the presence of a test compound than in the absence. For example, confluent cells plated in multi-well cell culture units are exposed to adenovirus luciferase or β-galactosidase constructs in the presence of the test compound. The test compound is administered to cells before, after or concurrently with the adenovirus. The extent of gene transfer is measured by visual inspection or quantitative measurement of luciferase or β-galactosidase in the presence and absence of the test compound. Compounds identified by this or similar assays as gene transfer activators are shown in Table I.

TABLE I

| Compound | Activity |
|---|---|
| 8496 | +++ |
| 6975 | +++ |
| 7064 | + |
| 6883 | + |
| 6898 | + |
| 7036 | ++ |

+++ = more active
++ = active
+ = less active

A gene transfer activator compound has the structure

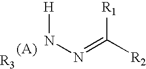

wherein R₁, R₂ and R₃ are each independently a C₀₋₁₂ substituent selected from the group consisting of: hydrogen, a heteroatom, alkyl, alkenyl, alkynyl, heteroatom substituted alkyl, heteroatom substituted alkenyl, heteroatom substituted alkynyl, aryl, arylalkyl, arylalkenyl, arylalkynyl with the proviso that at least one of R₁, R₂ and R₃ is not H; where the heteroatom is selected from the group consisting of: N, O and S; and where (A) is a single or double bond between N and R₃. The heteroatom is N, O or S. Further the C₀₋₁₂ substituent is linear, branched or cyclic and optionally includes a pendant moiety selected from the group consisting of: carbonyl, hydroxyl, carboxyl, amine, thiol, thioester, thioether, phosphate, alkoxy, aryl, arylalkyl, sulfonamide and alkyl halide.

Preferred compounds of the present invention are compound 6975 (A), compound 8496 (B), compound 6883 (C), compound 6898 (D), compound 7036 (E), and compound 7064 (F).

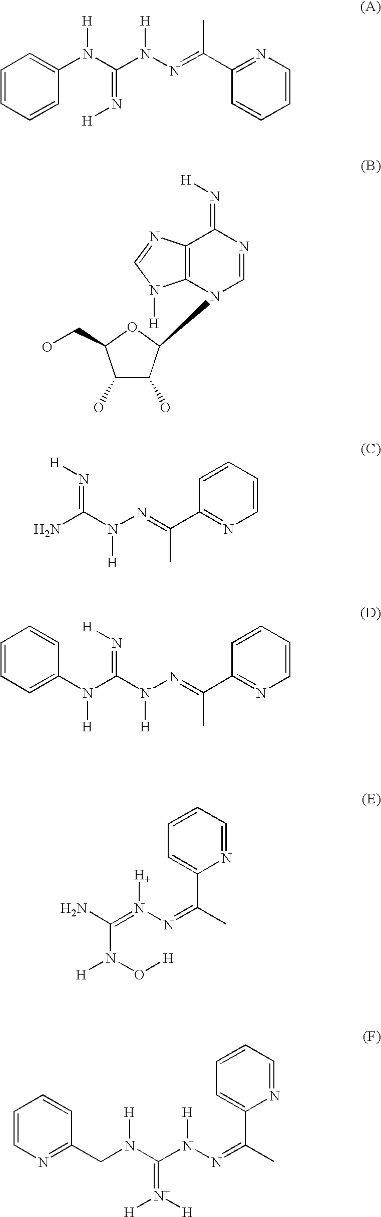

Methods of Treatment

The method of treatment basically consists of providing to cells the gene to be transferred and exposing the cells to at least one gene transfer activating compound. It will be apparent to one skilled in the art that multiple genes may be transferred. For example, more than one adenovirus construct or plasmid may be delivered to a cell or tissue. The gene to be transferred can be delivered directly to the targeted cells or tissue or administered systemically. In the latter case, the gene is delivered in combination with a targeting means, such as through the selection of a particular viral vector or delivery formulation. The gene transfer activating compound can also be administered directly to targeted cells or tissues, or systemically. Cells can be treated in vivo, within the patient to be treated, or treated ex vivo, then injected into the patient. Further, cells or tissue may be treated and maintained in vitro.

In some applications of a process provided by the present invention, cells that receive the recombinant vector are administered to the subject. A preferred embodiment of this process involves the ex vivo transfer of the gene to be expressed by incubation of the cells with the gene transfer construct and the gene transfer activating compound outside the body of the subject. The cells that receive the gene are introduced back into the subject where they express the therapeutic protein.

The route of gene transfer activating compound administration is oral, rectal, intraventricular, intracranial, intratumoral, intrathecal, intracisternal, intravaginal, parenteral, intravenous, intramuscular, subcutaneous, local, intraperitoneal, transdermal, by inhalation or as a buccal or nasal spray. The exact amount of gene transfer activating compounds required will vary from subject to subject, depending on the age, weight and general condition of the subject, the severity of the disease that is being treated, the location and size of the tumor, the particular compounds used, the mode of administration, and the like. An appropriate amount may be determined by one of ordinary skill in the art using only routine experimentation given the teachings herein.

The route of delivery of a pharmaceutically active amount of recombinant vector is oral, rectal, intraventricular, intracranial, intratumoral, intrathecal, intracisternal, intravaginal, parenteral, intravenous, intramuscular, subcutaneous, local, intraperitoneal, transdermal, by inhalation or as a buccal or nasal spray. The exact amount of recombinant vector required as a pharmaceutically active amount will vary from subject to subject, depending on the age, weight and general condition of the subject, the severity of the disease that is being treated, the location and size of the tumor, the particular compounds used, the mode of delivery, and the like. An appropriate amount may be determined by one of ordinary skill in the art using only routine experimentation given the teachings herein.

Depending on the intended mode of administration or delivery, the gene transfer activating compounds and recombinant vector can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. The compositions will include an effective amount of the selected gene transfer activating compounds in combination with a pharmaceutically acceptable carrier and, in addition, may include other medicinal agents, pharmaceutical agents, carriers, or diluents. By “pharmaceutically acceptable” is meant a material that is not biologically or otherwise undesirable, which can be administered to a subject along with the selected gene transfer activating compounds without causing significant undesirable biological effects or interacting in a deleterious manner with any of the other components of the pharmaceutical composition in which it is contained.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols, suitable mixtures thereof, vegetable oils and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, ethyl carbonate, ethyl acetate, benzyl alcohol, benzyl benzoate, propyleneglycol, 1,3-butyleneglycol, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the compositions can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate and laurylsulphonate salts, and the like. These may include cations based on the alkalai and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm. Sci., 1977; 66:1-19.

Examples of pharmaceutically acceptable, non-toxic esters of the compounds of this invention include C₁-C₆-alkyl esters wherein-the-alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines, and C₁-C₂ dialkyl secondary amines are preferred. Amides of the compounds of the invention may be prepared according to conventional methods.

The term “prodrug” refers to compounds that are rapidly transformed in vivo to yield the parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The compounds of the present invention can exist in different stereoisomeric forms by virtue of the presence of asymmetric centers in the compounds. It is contemplated that all stereoisomeric forms of the compounds, as well as mixtures thereof including racemic mixtures, form part of this invention.

The compounds of the present invention are administered to a subject at various dosage levels. The dosage depends on a number of factors illustratively including the size and age of the subject, the severity of the condition being treated, and the pharmacological activity of the compound being used. The determination of optimum dosages for a particular patient is well known to those skilled in the art.

In addition, it is intended that the present invention cover compounds made either using standard organic synthetic techniques, including combinatorial chemistry or by biological methods, such as through metabolism.

The present invention also includes a kit containing at least one of the compounds provided and can also include any reagents or components necessary for the administration of the compounds.

### EXAMPLES

The following examples are given for the purpose of illustrating various embodiments of the invention and are not meant to limit the present invention in any fashion.

### Example 1

HT29 cells are grown in 96-well plates. The cells are seeded at near confluency, about 300,000 cells per well, and infected with adenovirus 3-4 days later. At confluency on filters these tumor cells establish a resistance of 200-1000 Ω·cm² and exhibit vectoral chloride transport, two useful endpoints in evaluating viability. For screening purposes, cells are grown on plastic. Confluent monolayers under these conditions exhibit marked resistance to adenovirus entry. The activity of candidate compounds is also confirmed using polarized monolayers grown on filters. Once confluent, HT29 cells are very difficult to transduce with adenoviral constructs. Fewer than 1 in 100 cells express adenoviral transgenes at multiplicity of infection (MOI) of approximately 50. A replication deficient adenovirus encoding firefly luciferase or the E. coli LacZ gene (β-galactosidase) at an MOI of 50 for each well of cells in low serum medium is applied for 4 hours at 37° C. Test drugs are incubated along with the virus. Forty-eight hours later, the cells are assayed for β-galactosidase or luciferase by standard techniques. For example, β-galactosidase is assayed by incubating cells with a β-galactosidase substrate such as 1 mg/ml X-gal 5-bromo-4-chloro-3-indolyl B-D-galactopyranoside (X-gal), overnight at 37° C. When X-gal is cleaved by β-galactosidase, a bright blue precipitate is formed. The intensity of the blue color can be estimated visually or measured by optical densitometry to evaluate the efficiency of adenoviral transduction. Luciferase is assayed using a commercially available kit (Promega) which allows quantitative measurements of adenoviral gene transfer.

### Example 2

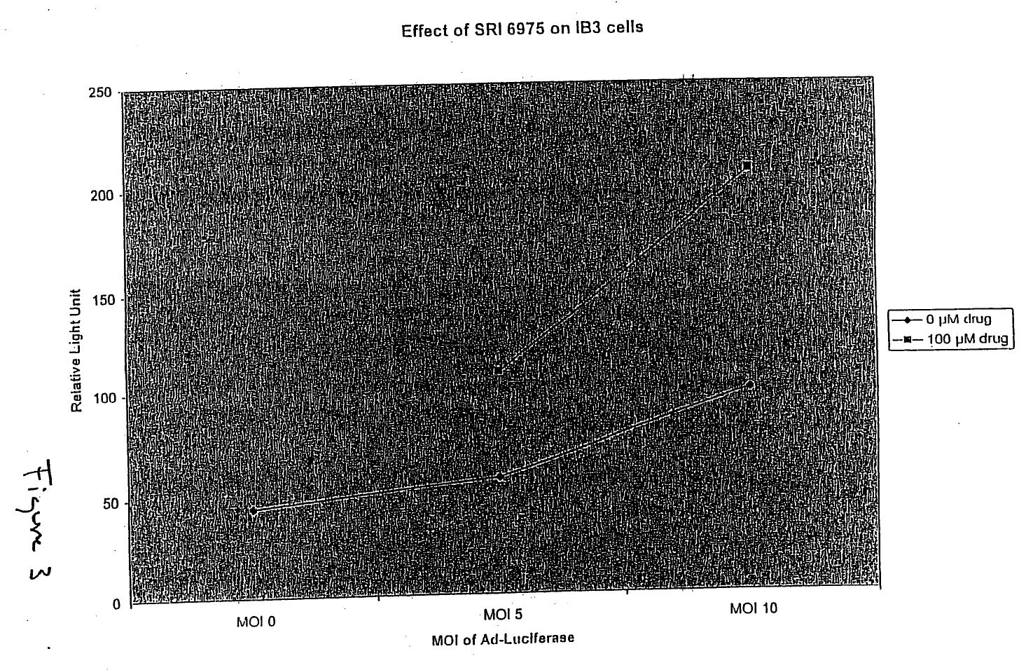

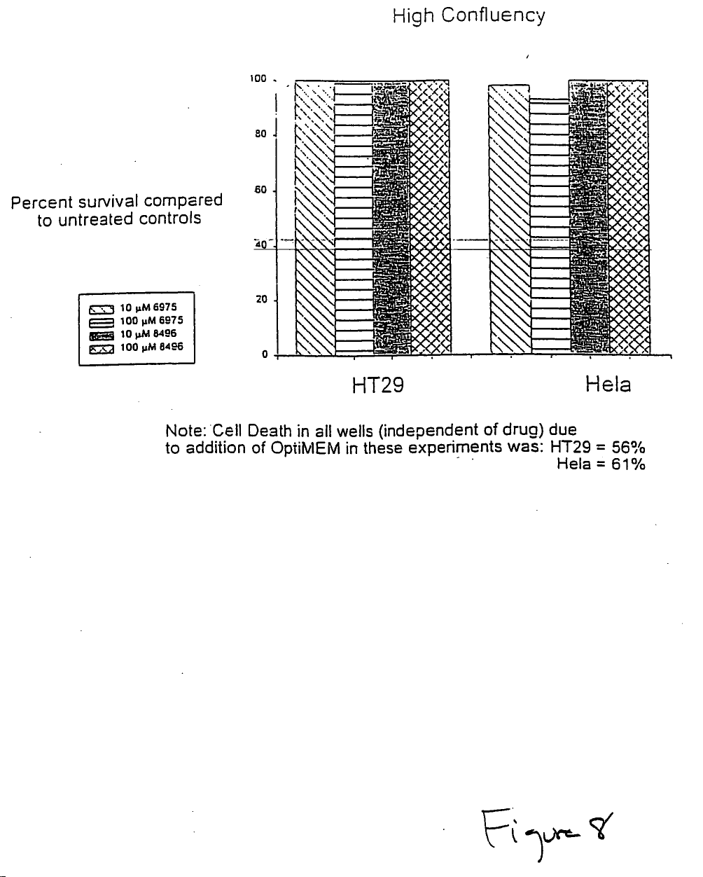

HT29 or HeLa cells are grown in 96-well plates. The HT29 cells are seeded at near confluency, about 300,000 cells per well, and infected with adenovirus 3-4 days later. Confluent HeLa cells are used as a positive control. Confluent monolayers under these conditions exhibit marked resistance to adenovirus entry. A replication deficient adenovirus encoding the E. coli LacZ gene is applied at an MOI of 0.1-10 to wells of cells in low serum medium for 4 hours at 37° C. Gene transfer activating compounds are incubated along with the virus at concentrations of 10 micromolar or 100 micromolar. A set of cells is incubated with virus but no gene transfer activating compound. Forty-eight hours later, the cells are assayed for β-galactosidase by standard techniques. Compounds 6975 and 8496 show activation of adenovirus gene transfer at both 10 and 100 micromolar in HT29 cells. FIGS. 2 and 6 illustrate the results using compound 6975 and 8496 respectively.

### Example 3

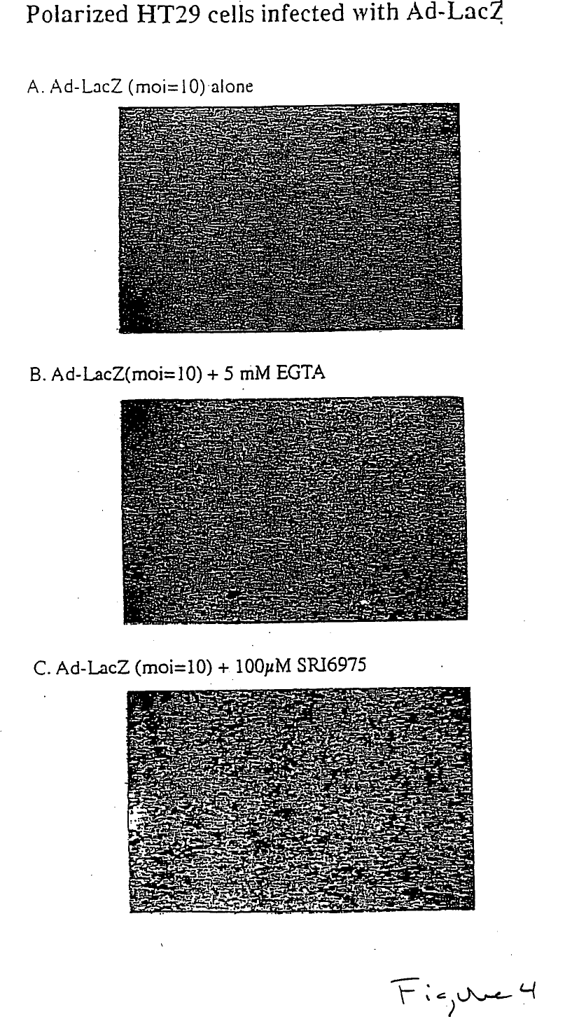

IB3 cystic fibrosis airway epithelial cells are grown in 96-well plates. The cells are seeded at near confluency, about 300,000 cells per well, and infected with adenovirus 3-4 days later. A replication deficient adenovirus encoding luciferase is applied at an MOI of 5 or 10 to wells of cells in low serum medium for 4 hours at 37° C. Compound 6975 is incubated along with the virus at a concentration of 100 micromolar. A set of cells is incubated with virus but no gene transfer activating compound. Forty-eight hours later, the cells are assayed for luciferase by standard techniques. FIG. 3 presents the results of this assay in a graph.

### Example 4

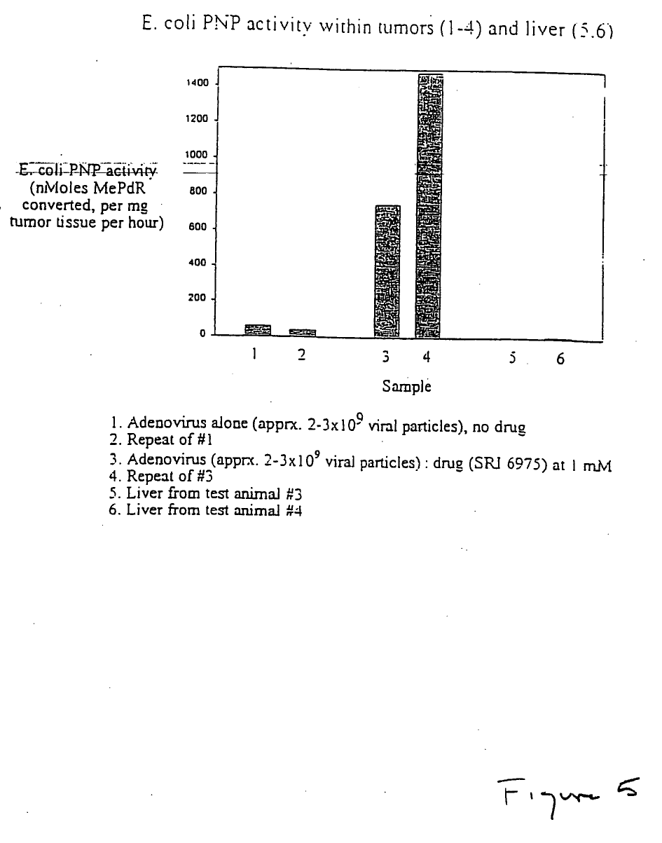

HT29 cells are grown in 96-well plates. The cells are seeded at near confluency, about 300,000 cells per well, and infected with adenovirus 3-4 days later. Confluent monolayers under these conditions exhibit marked resistance to adenovirus entry. A replication deficient adenovirus encoding LacZ is applied at an MOI of 10 to wells of cells in low serum medium for 4 hours at 37° C. Compound 6975 is incubated along with the virus at a concentration of 100 micromolar. A second set of cells is incubated with virus but no gene transfer activating compound and a third set of cells is incubated with virus and 5 millimolar EGTA. Forty-eight hours later, the cells are assayed for luciferase by standard techniques. Compound 6975 activates adenovirus gene transfer more efficiently than EGTA. FIG. 4 shows representative results of this assay.

### Example 5

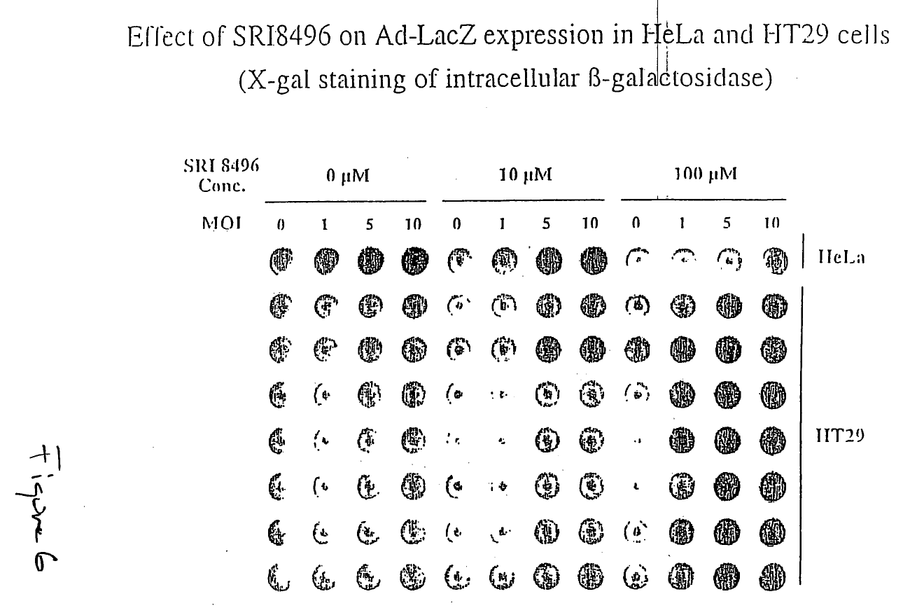

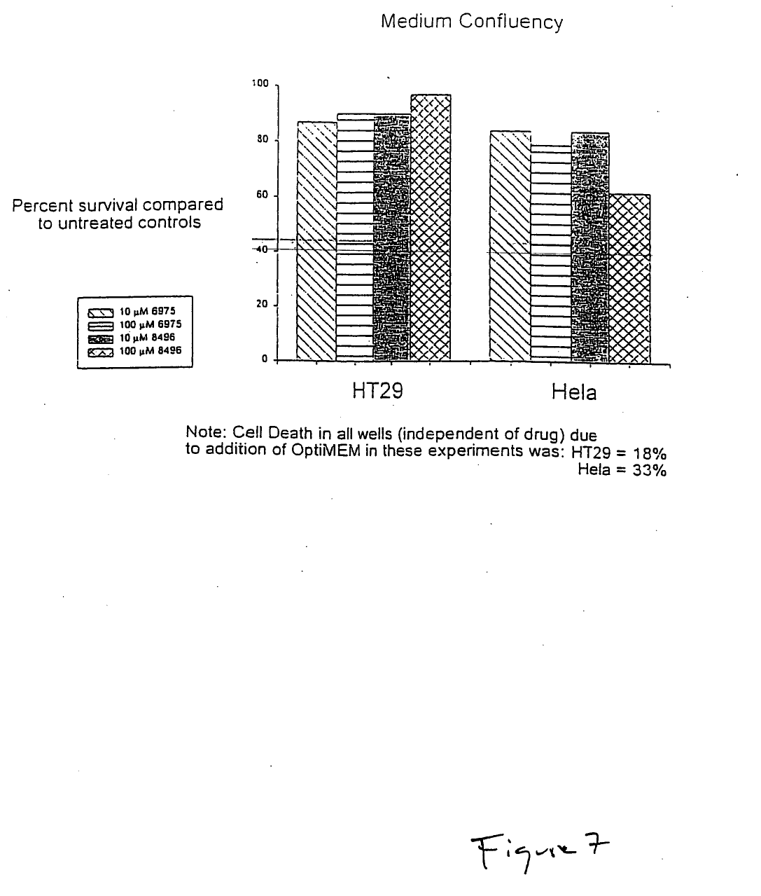

Prostate tumors are established in suitable hosts and PNP activity assayed in the presence or absence of adenovirus. Approximately 2-3×10⁹ adenoviral particles are administered per tumor in the presence of 1 mM of compound 6975 or in the absence of the compound. Transgene activity is also assayed in livers. Further details of the method are described in (3). FIG. 5 shows the results of these assays.

### Example 6

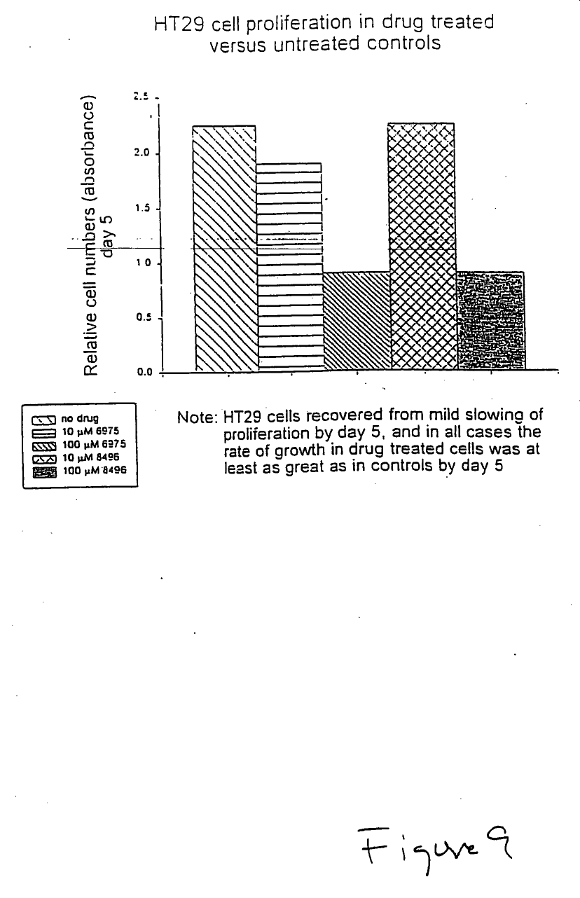

Gene transfer activating compounds 6975 and 8496 are assayed for effects on cell proliferation using HT29 and HeLa cells cultured at medium confluency. Each compound is added to achieve a final concentration of 10 micromolar or 100 micromolar. Measurements of cell proliferation are made according to the manufacturer's protocol using the Cytotox 96 non-radioactive assay which is commercially available from Promega Corp., Madison, Wis. FIG. 7 shows the effects of the gene transfer activating compounds 6975 and 8496 on cell proliferation at medium confluency.

### Example 7

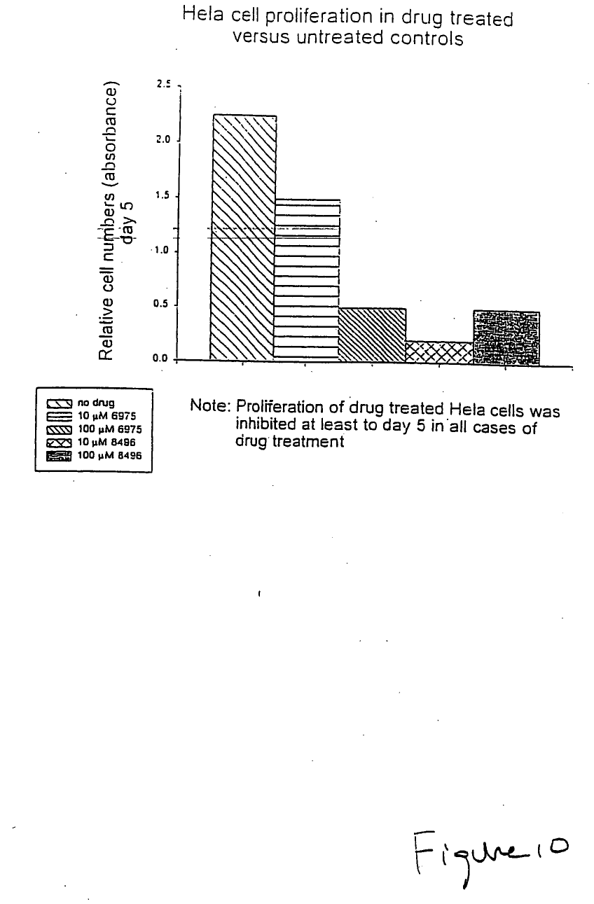

Gene transfer activating compounds 6975 and 8496 are assayed for effects on cell proliferation using HT29 and HeLa cells cultured at high confluency. Each compound is added to achieve a final concentration of 10 micromolar or 100 micromolar. Measurements of cell proliferation are made according to the manufacturer's protocol using the Cytotox 96 non-radioactive assay which is commercially available from Promega Corp., Madison, Wis. FIG. 8 shows the effects of the gene transfer activating compounds 6975 and 8496 on cell proliferation.

### Example 8

### Exemplary Synthesis of Semicarazones: Synthesis of Compound 6975.

To a solution of 1-phenyl-2-thiourea (1; 4.9 g; 32.03 mmole) in 75 mL anhydrous tetrahydrofuran solvent was added iodomethane (5 g; 35.23 mmole) and the mixture heated to reflux for 45 min. Anhydrous hydrazine (1 mL; 32.03 mmole) was then added. A white precipitate formed, which partially redissolved upon addition of ethanol (5 mL). The reaction mixture was reheated to reflux for 1 hr, then the solvent was removed under reduced pressure. Water (15 mL) was added to redissolve the resulting residue, whereupon a solution of silver nitrate (AgNO₃; 5.4 g) in 10 mL water was added. A yellow solid precipitate immediately formed. The reaction mixture was heated and filtered hot through Celite, and the Celite subsequently washed well with water. The combined filtrate and washes were evaporated under reduced pressure to a syrup, to which ethanol (5 mL) was added. Upon warming the material dissolved, and the solution was allowed to stand at 0° C. overnight. The resulting crystals were collected by filtration, and recrystallized from hot ethanol to afford pure intermediate phenylsemicarbazide 2.

To a suspension of 2 (2.1 g; 9.86 mmole) in ethanol (−100 mL) was added sodium methoxide (570 mg). The mixture was stirred for 5 min., then filtered through Celite. To the filtrate was added 2-acetylpyridine (1.2 mL) and the solution heated to reflux for 4 hr. Solvent was removed under reduced pressure, and the residue triturated with 100 mL ethyl ether. The white solid remaining after trituration was removed by filtration, and the filtrate was concentrated to −30 mL. Ethanolic HCl (1 M solution) was added until turbidity set in, and the solution was then allowed to stand at 4° C. overnight. The precipitate was collected and recrystallized from hot ethanol to yield pure SRI 6975.

### REFERENCES

1. Curlee K V, E J Sorscher. Design of gene therapy trials in CF patients. Cystic Fibrosis Methods and Protocols. Methods in Molecular Biology, 2001, In press.

2. Clancy J P, E J Sorscher. Liposomes for gene transfer Gene Therapy Technologies and Regulations: From Laboratory to Clinic, A. Meager, Ed., 1999.

3. Parker W B, S A King, P W Allan, L L Bennett Jr., J A Secrist III, J A Montgomery, K S Gilbert, W R Waud, A H Wells, G Y Gillespie, E J Sorscher. In vivo gene therapy of cancer with E. coli purine nucleoside phosphorylase. Human Gene Therapy 8:1637-1644, 1997.

4. Hughes B W, S A King, P W Allan, W B Parker, E J Sorscher. Cell to cell contact is not required for bystander cell killing by Escherichia coli purine nucleoside phosphorylase. J Bio. Chem. 273:2322-2328, 1998.

5. Zabner J, P Freimuth, A Puga, A Fabrega, M J Welsh. Lack of high affinity fiber receptor activity explains the resistance of ciliated airway epithelia to adenovirus infection. J Clin. Invest. 100:1144-9, 1997.

6. Grubb B R, R J Pickles, H Ye, J R Yankaskas, R N Vick, J F Engelhardt, J M Wilson, L G Johnson, R C Boucher. Inefficient gene transfer by adenovirus vector to cystic fibrosis airway epithelia of mice and humans. Nature 371:802-806.

7. Goldman M J, J M Wilson. Expression of αvβ integrin is necessary for efficient adenovirus-mediated gene transfer in the human airway. J Virology 69:5951-5958, 1995.

8. Myles C T, M Zhang, J C Kappes, Sorscher E J, Matalon S. Augmentation of adenovirus and lentivirus mediated gene transfer in lungs and epithelial cells by EGTA. Ped. Pulm. Suppl. 20:240 (Abst. 240), 2000.

9. Wang G, V Slepushkin, J Zabner, S Keshavjee, J C Johnston, S L Sauter, D J Jolly T W Dubensky, B L Davidson, P B McCray. Feline immunodeficiency virus vectors persistently transduce nondividing airway epithelia and correct the cystic fibrosis defect. J Clin. Invest. 104(11):R55-62, 1999.

10. Ziady A, T Ferkol. DNA condensation and receptor-mediated gene transfer. Gene Therapy Technologies, Applications and Regulations. Ed. A. Meager, 1999.

11. Gunzburg W H, B Salmons. Retroviral vectors. Gene Therapy Technologies, Applications and Regulations. Ed. A. Meager, 1999.

12. Lockett L J, Molloy P L, Russell P J, Both G W. Relative efficiency of tumor cell killing in vitro by two enzyme-prodrug systems delivered by identical adenovirus vectors. Clin. Cancer Res. 3:2075-2080, 1997.

13. Zufferery R, T Dull, R J Mandel, A Bukovsky, D Quiroz, L Naldini, D Trono. Self-inactivating lentivirus vector for safe and efficient in vivo gene delivery. J Virology 72(12):9873-80, 1998.

14. Lashford L S, L J Fairbaim, J E Wraith. Lysosomal storage disorders. Gene Therapy Technologies, Applications and Regulations. Ed. A. Meager, 1999.

15. Chamber R, G Y Gillespie, L Soroceanu, S Andreansky, S Chatterjee, J Chou, B Roizman, R J Whitley. Comparison of genetically engineered herpes simplex viruses for treatment of brain tumors in a scid mouse model of human malignant glioma. Proc. Natl. Acad. Sci USA 92:1411-1415, 1995.

16. Puhlmann M, M Gant, C K Brown, H R Alexander, D L Bartlett. Thymidine kinase-deleted vaccinia virus expressing purine nucleoside phosphorylase as a vector for tumor directed gene therapy. Human Gene Therapy 10:649-657, 1999.

17. Yamamoto K, R Morishita, N Tomita, T Shimozato, H Nakagami, A Kikuchi, M Aoki, J Higaki, Y Kaneda, T Ogihara. Ribozyme oligonucleotides against transforming growth factor-beta inhibited neointimal formation after vascular injury in rat model: Potential application of ribozyme strategy to treat cardiovascular disease. Circulation 102(11):1308-14, 2000.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A process for activating gene transfer comprising the steps of administering to a subject a pharmaceutically active amount of at least one gene transfer activating compound; and the step of delivering a pharmaceutically active amount of a recombinant vector comprising at least a nucleic acid such that said nucleic acid is transcribed in a target cell of said subject.

2. The process for activating gene transfer of claim 1 wherein said compound has the structure: 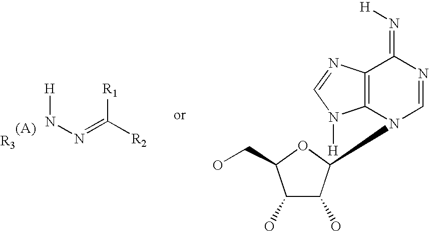
wherein R₁, R₂ and R₃ are each independently a C₀₋₁₂ substituent selected from the group consisting of: hydrogen, a heteroatom, alkyl, alkenyl, alkynyl, heteroatom substituted alkyl, heteroatom substituted alkenyl, heteroatom substituted alkynyl, aryl, arylalkyl, arylalkenyl, arylalkynyl with the proviso that at least one of R₁, R₂ and R₃ is not H; where the heteroatom is selected from the group consisting of: N, O and S; and where (A) is a single or double bond between N and R₃.

3. The process for activating gene transfer of claim 2 wherein the C₀₋₁₂ substituent is selected from the group consisting of linear, branched and cyclic.

4. The process for activating gene transfer of claim 2 where any C, N, O or S in the C₀₋₁₂ substituent has a pendant moiety selected from the group consisting of: carbonyl, hydroxyl, carboxyl, amine, thiol, thioester, thioether, amide, phosphate, alkoxy, aryl, arylalkyl, sulfonamide and alkyl halide.

5. The process for activating gene transfer of claim 1 wherein said compound is selected from the group consisting of: 6883, 6898, 6975, 7036, 7064 and 8496.

6. The process for activating gene transfer of claim 1 wherein the administration is by a route selected from the group consisting of: oral, rectal, parenteral, intravenous, intramuscular, subcutaneous, intracisternal, intravaginal, intraperitoneal, intravesical, intraventricular, intracranial, intratumoral, local, transdermal, intrabuccal, intranasal and intrathecal.

7. The process for activating gene transfer of claim 1 wherein the delivery is by a route selected from the group consisting of: oral, rectal, parenteral, intravenous, intramuscular, subcutaneous, intracisternal, intravaginal, intraperitoneal, intravesical, intraventricular, intracranial, intratumoral, local, transdermal, intrabuccal, intranasal and intrathecal.

8. The process for activating gene transfer of claim 1 wherein said target cell is an epithelial cell.

9. The process for activating gene transfer of claim 1 wherein said recombinant vector is a virus.

10. The process for activating gene transfer of claim 9 wherein said virus is adenovirus.

11. The process for activating gene transfer of claim 9 wherein said virus is selected from the group consisting of: lentivirus, adeno-associated virus, retrovirus, vaccinia virus, and herpes simplex virus.

12. The process for activating gene transfer of claim 1 wherein said recombinant vector is a plasmid.

13. The process for activating gene transfer of claim 1 wherein said subject is selected from the group consisting of: human, cow, horse, sheep, pig, goat, chicken, cat, dog, mouse and rat.

14. The process for activating gene transfer of claim 1 wherein said subject has a pathological condition.

15. The process for activating gene transfer of claim 14 wherein said pathological condition is associated with a lacking or mutant-protein.

16. The process for activating gene transfer of claim 15 wherein said pathological condition is selected from the group consisting of: galactosemia, phenylketonuria, Duchenne muscular dystrophy, Lesh-Nyhan syndrome, severe combined immunodeficiency syndrome, thalassemia, sickle cell anemia, cystic fibrosis, a₁ antitrypsin deficiency, cancer, lysosomal storage disorders, porphyria and hemophilia.

17. The process for activating gene transfer of claim 14 wherein said pathological condition is growth of abnormal cells.

18. The process for activating gene transfer of claim 17 wherein said abnormal cells are cancer cells.

19. The process for activating gene transfer of claim 14 wherein said pathological condition is virus infection.

20. The process for activating gene transfer of claim 1 wherein said nucleic acid comprises at least a protein encoding sequence such that the nucleic acid is transcribed and translated in the target cell.

21. The process for activating gene transfer of claim 20 wherein said protein is selected from the group consisting of: mammalian and non-mammalian.

22. The process for activating gene transfer of claim 20 wherein said protein is a marker.

23. The process for activating gene transfer of claim 22 wherein said marker is selected from the group consisting of: green fluorescent protein, luciferase and β-galactosidase.

24. A compound having the structure: 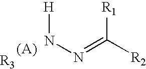
wherein R₁, R₂ and R₃ are each independently a C₀₋₁₂ substituent selected from the group consisting of: hydrogen, a heteroatom, alkyl, alkenyl, alkynyl, heteroatom substituted alkyl, heteroatom substituted alkenyl, heteroatom substituted alkynyl, aryl, arylalkyl, arylalkenyl, arylalkynyl with the proviso that at least one of R₁, R₂ and R₃ is not H; where the heteroatom is selected from the group consisting of: N, O and S; and where (A) is a single or double bond between N and R₃.

25. The compound of claim 24 wherein the heteroatom is selected from the group consisting of: N, O and S.

26. The compound of claim 24 wherein the C₀₋₁₂ substituent is selected from the group consisting of linear, branched and cyclic.

27. The compound of claim 24 where any atom in the C₀₋₁₂ substituent has a pendant moiety selected from the group consisting of: carbonyl, hydroxyl, carboxyl, amine, thiol, thioester, thioether, phosphate, alkoxy, aryl, arylalkyl, sulfonamide and alkyl halide.

28. A process for activating gene transfer comprising the steps of administering to a cell a compound selected from the group consisting of: 6883, 6898, 6975, 7036, 7064, 8496 and a compound according to claim 24; and delivering a recombinant vector comprising at least a nucleic acid such that said nucleic acid is transcribed in said cell.

29. The process for activating gene transfer of claim 28 wherein said cell is an epithelial cell.

30. The process for activating gene transfer of claim 28 wherein said recombinant vector is a virus.

31. The process for activating gene transfer of claim 30 wherein said virus is adenovirus.

32. The process for activating gene transfer of claim 30 wherein said virus is selected from the group consisting of: lentivirus, adeno-associated virus, retrovirus, vaccinia virus, and herpes simplex virus.

33. The process for activating gene transfer of claim 28 wherein said recombinant vector is a plasmid.

34. The process for activating gene transfer of claim 28 wherein said cell is derived from a subject from the group consisting of: human, cow, horse, sheep, pig, goat, chicken, cat, dog, mouse and rat.

35. The process for activating gene transfer of claim 34 wherein said subject has a pathological condition.

36. The process for activating gene transfer of claim 35 wherein said pathological condition is associated with a lacking or mutant protein.

37. The process for activating gene transfer of claim 35 wherein said pathological condition is selected from the group consisting of: galactosemia, phenylketonuria, Duchenne muscular dystrophy, Lesh-Nyhan syndrome, severe combined immunodeficiency syndrome, thalassemia, sickle cell anemia, cystic fibrosis, a₁ antitrypsin deficiency, cancer, lysosomal storage disorders, porphyria and hemophilia.

38. The process for activating gene transfer of claim 35 wherein said pathological condition is growth of abnormal cells.

39. The process for activating gene transfer of claim 38 wherein said abnormal cells are cancer cells.

40. The process for activating gene transfer of claim 28 wherein said cell is in vitro.

41. The process for activating gene transfer of claim 28 wherein said cell is ex vivo.

42. The process for activating gene transfer of claim 28 wherein said cell is in vivo.

43. A compound having the structure:

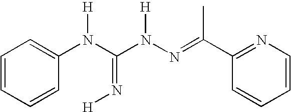

44. A compound having the structure:

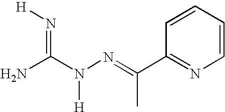

45. A compound having the structure:

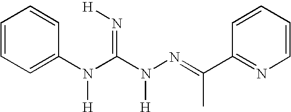

46. A compound having the structure:

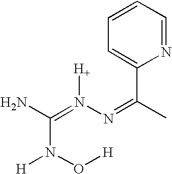

47. A compound having the structure:

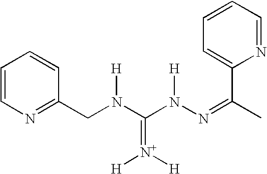

48. A kit for activating gene transfer comprising a compound selected from the group consisting of: 6883, 6898, 6975, 7036, 7064, 8496 and a compound according to claim 39; packaged in a suitable container together with instructions for use.
